(19) United States
(12) Patent Application Publication
(10) Pub. No.: US 2023/0012667 A1
(43) Pub. Date: Jan. 19, 2023

(54) DISINFECTING CONTAINER

(71) Applicant: Virastrike Holdings Pty Ltd, Brisbane (AU)

(72) Inventors: Simon ASIMUS, Brisbane (AU); Will SMART, Brisbane (AU)

(21) Appl. No.: 17/848,066

(22) Filed: Jun. 23, 2022

(30) Foreign Application Priority Data
Jun. 24, 2021 (AU) 2021901912

(51) Int. Cl. A61L 2/10; A61L 2/26

(52) U.S. Cl. CPC A61L 2/10; A61L 2/26; A61L 2202/11

(57) ABSTRACT

Disclosed herein is a disinfecting container for a good or piece of equipment. The disinfecting container includes a body in which to place the good or piece of equipment. The body has an internal recess that has an interior reflective surface. The disinfecting cabinet also includes a cover that is attached or attachable to the body. There is at least one UV emitting light within the container. The disinfecting container also includes a control system that is operatively associated with a power source to turn on or off the at least one UV emitting light. The UV emitting light emits UV light at a wave length and for a predetermined period to achieve substantial disinfection of the good or piece of equipment.

### FIELD

The present disclosure relates to a disinfecting container and method of using the disinfecting container for reducing contamination by bacteria, viruses and other microorganisms on the surface of an object. The present disclosure has particular but not exclusive application for disinfecting personal equipment. In the specification the disinfection of sporting equipment will be described by way of example only and embodiments of the disclosure are not limited to this example.

### BACKGROUND

Personal equipment or goods can be contaminated with bacterial and or viral pathogens though physical contact with people and animals and exposure in contaminated environments. For example, the surface of the sporting equipment and goods can be contaminated by the respiratory droplets from breathing or sweating from an infected person. The contaminated goods or equipment can cause infection by acting as a vector for transmitting bacteria and virus through human-to-human contact. Equipment or goods that may become contaminated include but are not limited to sporting goods, exercise equipment, toys, tools, medical apparatus and personal hygiene products.

Equipment and goods are currently disinfected by wiping with a cloth containing 70% isopropyl alcohol or 0.2% w/w benzalkonium. The equipment is then rinsed with water and left to dry at room temperature for 2 hours. However, the efficiency of disinfection is subject to human error when performing the wiping. In addition, it requires a long disinfection time and a time consuming procedure before the equipment can be used again. Further, residual chemicals and or water due to incomplete drying can remain on the surface creating a biofilm environment and make the surface of the equipment.

Disinfection failure results in residual bacteria and viruses remaining on the surface of the equipment and goods. Using contaminated equipment or goods that contains bacteria and microorganisms can lead to hand-to-hand transmission of infectious microorganisms.

### SUMMARY

It is an objective of embodiments of the present invention to provide an alternative means for disinfecting goods and equipment and a method of using such means which overcomes or ameliorates one or more of the disadvantages or problems described above, or which at least provides a useful alternative.

Embodiments of the present disclosure were developed from the need to disinfect personal equipment that is thorough and fast so that the equipment can be quickly reused. The inventors took an alternate approach to the current method of wiping equipment with anti-microbial wipes. The inventors' aim was to make the method quick so the turnaround time or the time before the goods or equipment could be reused is greatly reduced and to reduce human error by making the method reproducible irrespective of who performed the disinfection. That is, an aim was to reduce or eliminate human error as much as possible in the method of disinfecting equipment.

The inventors experimented with different types of physical treatment of the good and equipment. However, they found that there was limited efficiency in successful disinfection with physical treatment. In addition, they found that the physical application of disinfectant could affect and potentially damage the outer material of the good or equipment.

Consequently, the inventors took an alternative approach by investigating disinfection treatment of personal equipment using Ultraviolet (UV) light. They experimented and trialed with different UV light sources, wavelengths and intensities, size of the treatment containers and the position of the UV light sources within the treatment container. The inventors determined the UV light wave length, the number of the UV lights and the position of the UV lights in the container that can provide a fast and effective disinfecting treatment to the surface of the personal equipment.

The inventors have considered that application of at least one embodiment of the disclosure is suited in a variety of fields requiring portable disinfection technology including but not limited to the medical industry, aged care, childcare, veterinary care, sport, customs services, quarantine services, postal services, food services, and personal use. Application of at least one embodiment of the disclosure can be of particular use during humanitarian disasters.

At least one aspect of the present disclosure broadly resides in a disinfecting container for treating a good or piece of equipment, the container including:
a body in which to place the good or piece of equipment, the body has an internal recess and the internal recess has an interior reflective surface;
a cover that is attached or attachable to the body;
at least one UV emitting light within the container; and
at least one control means that is operatively associated with a power source, the control means is configured to turn on or off the at least one UV emitting light,
wherein the UV emitting light emits UV light at a wavelength and for a predetermined period to achieve substantial disinfection on the surface of the good or piece of equipment.

Another aspect of the present disclosure broadly resides in a disinfecting container for treating a good or piece of equipment, the container including:
a body in which to place the good or piece of equipment, the body has an internal recess and the internal recess has an interior reflective surface;
a cover that is attached or attachable to the body;
at least one UV emitting light within the container; and
at least one control system that is operatively associated with a power source, the control system is configured to turn on or off the at least one UV emitting light,
wherein the UV emitting light emits UV at a wavelength and for a predetermined period to achieve disinfection on a surface of the good or piece of equipment.

The at least one UV emitting light preferably emits UV light that has a wavelength in the range of substantially 100 to 400 nm, more preferably in the range of substantially 100 to 280 nm, more preferably in the range of substantially 200 to 280 nm and most preferably in the range of substantially 250 to 265 nm. The at least one UV emitting light preferably emits UV light that has a wavelength of substantially 265 nm.

In one embodiment, the disinfecting container emits multiple wavelengths simultaneously, in sequence or in a pattern to achieve substantial disinfection on the surface of the good or piece of equipment. Utilising multiple wavelengths can increase the disinfection percentage. In one embodiment the disinfecting container uses at least one UV emitting light that emits UV light that has a wavelength of substantially 250 nm and at least one UV emitting light that emits UV light that has a wavelength of substantially 265 nm.

In a first preferred embodiment, the at least one UV emitting light is positioned within the body of the container. In a second preferred embodiment, the at least one UV emitting light is positioned at the cover. In a third preferred embodiment, at least one UV emitting light is positioned at the cover and at least one UV emitting light is positioned within the body of the container.

Preferably, the container includes a plurality of UV emitting lights. Preferably, the plurality of UV emitting lights are arranged and positioned in a pattern that substantially irradiates the entire surface or exposed surface of the good or piece of equipment placed within the container.

In a preferred embodiment the disinfecting container has an array of UV emitting lights wherein:
each UV emitting light has an angle of emission of substantially 120 degrees;
each UV emitting light is arranged so that the emitted light travels within a range of 40 mm to 140 mm from the UV emitting light to the object; and
the arrangement of array of UV emitting lights achieves a UV radiation intensity of at least 4.5 mJ/cm² on the exposed surface of the good or piece of equipment placed within the container.

The UV emitting light preferably is a UV light-emitting diode (LED) light.

The control system preferably includes a display and a control relay.

The display preferably is a digital display. The digital display preferably functions as a touchscreen, wherein the digital display includes a touch panel and a display panel. The touch panel preferably turns on or off the UV emitting light. The display panel preferably displays the time remaining to complete a disinfection cycle.

The control relay preferably controls the at least one UV emitting light. The control relay preferably is in communication with the touch panel to turn on or off the UV emitting light. The control relay preferably further includes a safety switch. The safety switch preferably turns off the UV emitting light when the cover is displaced from the container.

In a first preferred embodiment, the control system preferably further includes at least one LED indicator to indicate whether or not there is sufficient power from the power source. In a second preferred embodiment, the display presents a status of the power source including whether there is sufficient power from the power source and or the amount of power remaining from the power source.

The UV emitting light is preferably powered by at least one power source. The at least one power source preferably also provide power to the display. The at least one power source preferably includes at least one internal battery, at least one external battery, at least one uninterruptible power supply (UPS) battery, a solar power panel, an AC power source and or a DC power source. Preferably, the disinfecting container further includes a connector to connect to the power source. The connector preferably is a power cable which connects the disinfecting container to an AC or DC power source.

The control system preferably automates a disinfection cycle.

Automation preferably includes at least activating the UV emitting lights and deactivating the UV emitting lights after a predetermined period.

The control system preferably further includes a motion sensor to record hand gestures as control inputs. Preferably the use of gestures are employed as control inputs thereby reducing the need to physically touch the device and thus reduce the possibility to cross-contamination.

The control system preferably controls an actuator, wherein the actuator is adapted to remove or place a cover, remove or place an object in the container, and/or reorient an object within the container. The actuator reduces the need to physically touch the device and thus reduces the possibility to cross-contamination.

The body preferably further includes at least one liner that covers at least one inner surface of the body. Preferably, there is substantial space between the at least one liner and the at least one inner surface of the body that allows air to flow through. In a first preferred embodiment, the at least one liner preferably has at least one side that is formed by a reflective material. In a second preferred embodiment the at least one liner preferably is made entirely from a reflective material. The at least one liner preferably forms the interior reflective surface of the body. Preferably, the reflective material is stainless steel. Preferably, the inner recess is formed by a plurality of the liners that cover the inner surface of the body.

The cover preferably further includes at least one liner provided on one side of the cover. Preferably, the liner is provided on the side of the cover facing the interior of the body. Preferably, there is substantial space between the at least one liner and the side of the cover that allows air to flow through. Preferably, the at least one liner forms a reflective surface on the cover.

Preferably, the at least one UV emitting lights is mounted to the at least one liner. Preferably, the at least one UV emitting lights is mounted to the at least one liner by a mounting frame. Preferably, the mounting frame includes at least one pocket that houses the at least one UV emitting lights and at least two mounting openings. The at least one pocket preferably includes a mounting aperture for attaching or detaching the at least one UV emitting lights. Preferably, each of the at least two mounting openings receives a fastener for mounting to the at least one liner. Preferably, the at least one liner includes a plurality of first openings to accommodate the UV emitting lights. Preferably, the at least one liner includes a plurality of second openings to receive the fastener that passes through the mounting openings of the mounting frame.

In one embodiment at least one UV emitting light is mounted on a liner removably fitted to the container. Preferably the removable liner can be repositioned within or removed from the container to accommodate use of the container with regard to differently sized and/or shaped objects to be disinfected.

The disinfecting container preferably further includes a UV sensor. The UV sensor preferably gathers readings of the UV activity within the container during a disinfection cycle. The control system preferably varies the duration of UV light exposure required to complete a disinfection cycle based on the UV readings of the UV sensor. For example, if the readings indicate the UV exposure activity within the container is less than what is required for disinfection the control system can increase the duration of the disinfection cycle to compensate.

The disinfecting container preferably further includes at least one air inlet and at least one air outlet. Preferably, the at least one air inlet and at least one air outlet allows air flow through the container. The disinfecting container preferably further includes at least one filter. Preferably, the at least one filter is filters out particles from air passing through the filter. The disinfecting container preferably includes at least one air inlet, at least one air outlet, and one or more extractors to create an air flow within the disinfecting container, and the at least one air inlet and or the at least one air outlet has at least one air filter to filter particles or microorganisms.

In a first preferred embodiment, the at least one air inlet includes a filter filters air passing through the air inlet. In a second preferred embodiment, the at least one outlet includes a filter filters air passing through the air outlet. In a third preferred embodiment, the at least one air inlet and at least one air outlet each includes a filter. The at least one filter preferably is a high-efficiency particulate air (HEPA) filter.

Preferably, the disinfecting container further includes one or more extractors to create an air flow within the disinfecting container. In a first preferred embodiment, the one or more extractors are located in the body of the disinfecting container. In a second preferred embodiment, the one or more extractors are located in the cover. In a third preferred embodiment, at least one extractor is located in the body and at least one extractor is located in the cover. The extractor preferably is an exhaust fan.

Preferably, the extractor can reduce heat generated from the UV emitting lights in the container by creating an airflow within the container. Preferably, the extractor can remove heat generated from any electronic components within the container by creating an airflow within the container.

Contaminated wet or moist objects carry the risk of microbiological proliferation making disinfection less effective. It is preferred that a contaminated object is dry before performing a disinfection cycle.

Preferably, the extractor can substantially dry any wet object placed within the container by creating an airflow through the container. Preferably, the extractor can substantially dry a surface of an object placed within the container by creating an airflow through the container. Preferably, the extractor can draw air through an air inlet of the container, through the container and then through an air outlet of the container to create an air flow through the container. Preferably the extractor performs a drying cycle independently of the disinfection cycle.

Preferably, the container further includes a dehumidifier to substantially dry any wet object placed within the container.

Preferably, the disinfecting container further includes a heat sink to reduce heat generated from the UV emitting lights in the container.

In one embodiment, the disinfecting container has a first extractor that removes heat generated from any electronic components within the container by creating an airflow within the container and a second extractor that substantially dries any wet object placed within the container by creating an airflow through the container. Preferably the body has a volume situated behind the internal recess, the volume substantially housing any electronic components within the container wherein the first extractor creates an airflow through the volume. More preferably the second extractor creates an airflow through the internal recess.

In a first preferred embodiment, the disinfecting container further includes at least one support that holds the good or piece of equipment in position within the body. The at least support preferably holds the good or piece of equipment up from the bottom of the body. Preferably, the at least one support is a rack.

In a second preferred embodiment, the disinfecting container further includes at least one manipulator within the body where the good or piece of equipment is placed or positioned. Preferably, the manipulator to move and expose all outer surfaces of the good or piece of equipment to the UV during disinfection. Preferably, the at least one manipulator is a conveyor system or a rotating mesh drum.

The disinfecting container preferably further includes one or more locks to seal or lock the container.

The disinfecting container preferably has at least one roller wheel attached to a base portion of the body of the container. Preferably, the body has one or more swivel-mounted wheels to provide maneuverability.

The disinfecting container preferably can be any suitable size. The disinfecting container preferably can be purpose-built to a size that is suitable for a particular good or piece of equipment. The disinfecting container preferably has a capacity of 1 to 120 litres, and more preferably of 10 to 70 litres. In a first preferred embodiment, the interior of the disinfecting container has a dimension of 420 mm×330 mm×180 mm. In a second preferred embodiment, the interior disinfecting container has a dimension of 700 mm×500 mm×400 mm. In an alternative embodiment, the interior of the disinfecting container is substantially cylindrical with an interior dimension of 0130 mm×200 mm.

The disinfecting container preferably is portable. Preferably, the body further includes at least one handle. Preferably, the cover further includes at least one handle. Preferably the body and the cover each includes at least one handle.

The good or piece of equipment preferably is a sporting good or sporting equipment. In one embodiment, the sporting equipment is a ball for sports. The ball for sports preferably includes but not limited to a football or soccer ball, baseball, basketball, billiard ball, golf ball, volleyball, rugby ball, bowling ball, lacrosse ball, cricket ball, water polo ball, tennis ball, and table tennis ball.

Another aspect the present disclosure broadly resides in a system for disinfecting a good or piece of equipment including
a disinfecting container as herein described,
a support as herein described and positioned within the disinfecting container to support the good or piece of equipment, and
at least one extractor as herein described to create an airflow within the container,
wherein the good or piece of equipment is placed within the container and when the container is closed, the good or piece of equipment is exposed to the UV lights at a pre-determined period of time to achieve substantial disinfection.

Another aspect of the present disclosure broadly resides in a system for disinfecting a good or piece of equipment including
a disinfecting container as herein described,
a manipulator as herein described and positioned within the disinfecting container where the good or piece of equipment is placed or positioned, and
at least one extractor as herein described to create an airflow within the container,
wherein the good or piece of equipment is placed within the container and when the container is closed, the good or piece of equipment is exposed to the UV lights at a pre-determined period of irradiation time to achieve substantial disinfection.

Preferably, substantial disinfection is achieved where substantially 70% or more of pathogens are eliminated from the surface of the good or piece of equipment and more preferably where substantially 90% or more of pathogens are eliminated from the surface of the good or piece of equipment.

Preferably, substantial disinfection is determined by a cumulative light amount of UV lights or UV dose shone on the surface of the good or piece of equipment. Preferably, the cumulative light amount of UV or the UV does is determined by the intensity of the UV lights on the surface of the good or piece of equipment times the pre-determined irradiation time.

Preferably, the intensity of the UV lights on the surface of the good or piece of equipment is in the range of substantially 0.01 to 1 mW/cm², more preferably in the range of substantially 0.04 to 0.2 mW/cm², and most preferably in the range of substantially 0.05 to 0.1 mW/cm².

Preferably, the pre-determined irradiation time to treat viral contamination is in the range of substantially 10 to 150 seconds, more preferably in the range of substantially 20 to 100 seconds, and most preferably in the range of substantially 45 to 90 seconds.

Preferably, the pre-determined irradiation time to treat bacterial contamination is in the range of substantially 200 to 400 seconds, more preferably in the range of substantially 230 to 310 seconds, and most preferably in the range of substantially 240 to 300 seconds.

Preferably, the interior reflective surface of the container formed by the at least one liner reflects the UV radiation emitted from the UV emitting lights so that the entire surface or exposed surface of the good or piece of equipment is substantially irradiated.

Preferably, the entire surface or exposed surface of the good or piece of equipment is substantially irradiated by a combination of direct emissions from the UV emitting lights and radiation reflected from a reflective surface. More preferably, the container further includes a diffuser to diffuse UV light to increase the variety of incident ray angles striking the good or piece of equipment. Some surfaces are complex, such as hook and loop fasteners, and parts of the surface may block other parts of the surface from receiving UV light causing ‘shadows’. Increasing the variety of incident ray angles striking the good or piece of equipment can reduce the formation of shadows. This can be achieved by having a sufficient quantity of UV emitting lights, utilising reflection and/or diffusion to increases the variety of incident ray angles, and/or reorienting the object or good within the container.

Preferably, the control system of the disinfecting container turns on the UV emitting lights at the beginning of the disinfection and turn off the UV emitting lights after the predetermined time.

Another aspect the present disclosure broadly resides in a method of disinfecting a good or piece of equipment, the method including
placing the good or piece of equipment into a disinfecting container as herein described,
closing a cover on the container,
turning on the UV emitting lights,
exposing the good or piece of equipment to the UV emitting lights at a predetermined time to achieve substantial disinfection,
turning off the UV emitting lights after the predetermined time,
opening the cover on the container, and
removing the good or piece of equipment from the container.

In a first preferred embodiment, the step of placing the good or piece of equipment into the container includes placing the good or piece of equipment to a support as herein described within the container that holds the good or piece of equipment in position during the disinfecting process. Preferably, the support holds the good or piece of equipment up from the bottom of the body to increase the outer surface area that is exposed to the UV emitting light.

In a second preferred embodiment, the step of placing the good or piece of equipment into the container includes placing the good or piece of equipment to a manipulator as herein described within container. Preferably, the manipulator moves the good or piece of equipment and exposes all outer surfaces of the good or piece of equipment to the UV during the disinfection process.

Preferably, there is a step of closing the cover on the container further including locking the container to prevent accidental displacement of the cover.

Preferably, prior to the step of opening the cover, the method further includes a step of unlocking the container.

A further aspect of the present disclosure broadly resides in a disinfecting container for treating a good or piece of equipment, the container including:
a body in which to place the good or piece of equipment, the body has an internal recess and the internal recess has an interior reflective surface;
a cover that is attached or attachable to the body;
at least one array of UV emitting lights within the container; and
at least one control system that is operatively associated with a power source, the control system is configured to turn on or off the at least one array of UV emitting lights,
   wherein the at least one array of UV emitting lights emits UV light at a wave length in the range of substantially 250 to 265 nm
   wherein each UV emitting light has an angle of emission of substantially 120 degrees;
   wherein each UV emitting light is arranged so that the emitted light travels substantially within a range of 40 mm to 140 mm from the UV emitting light to the object; and
   wherein the arrangement of array of UV emitting lights achieves a UV radiation intensity of at least 4.5 mJ/cm² on the exposed surface of the good or piece of equipment placed within the container; and for a predetermined period to achieve substantial disinfection on the surface of the good or piece of equipment.

The features described with respect to one aspect also apply where applicable to all other aspects recited in the disclosure. Furthermore, different combinations of described features are herein described and claimed even when not expressly stated.

### BRIEF DESCRIPTION OF THE DRAWINGS

In order that the present invention can be more readily understood reference will now be made to the accompanying drawings which illustrate embodiments of the disclosure and wherein:

FIG. 1 is a three-dimensional (3D) diagrammatic view of a disinfecting container of the preferred embodiment in use;

FIG. 2 is a 3D diagrammatic view of the disinfecting container of FIG. 1 with a closed cover;

FIG. 3 is a cross-sectional side view of the disinfecting container of FIG. 2 in use;

FIG. 4 is a cross-sectional top view of a body of the disinfecting container of FIG. 1;

FIG. 5 is a cross-sectional bottom view of a cover of the disinfecting container of FIG. 1;

FIG. 6 is a 3D diagrammatic view of a mounting LED strip of the preferred embodiment;

FIG. 7 is a front view of the mounting LED strip of FIG. 6 and FIG. 8 is a diagrammatic view of the cross-section A-A of FIG. 7;

FIG. 9 is a 3D diagrammatic back view of a reflective liner of the preferred embodiment with the mounting LED strip of FIG. 6, and FIG. 10 is the front view of the reflective lining of FIG. 9;

FIG. 11 is a 3D diagrammatic view of a disinfecting container of the further embodiment in use;

FIG. 12 is a top view of the disinfecting container of FIG. 10;

FIG. 13 is a cross-sectional side view B-B of FIG. 11.

FIG. 14 is a cross-sectional side view C-C of FIG. 11.

FIG. 15 is a side view of detail Z of FIG. 13.

FIG. 16 is a side view of detail Y of FIG. 13.

### DETAILED DESCRIPTION

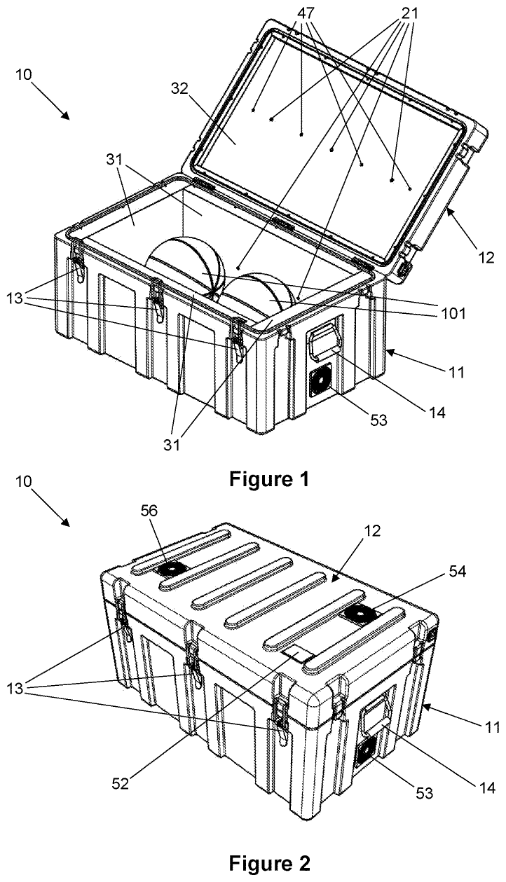

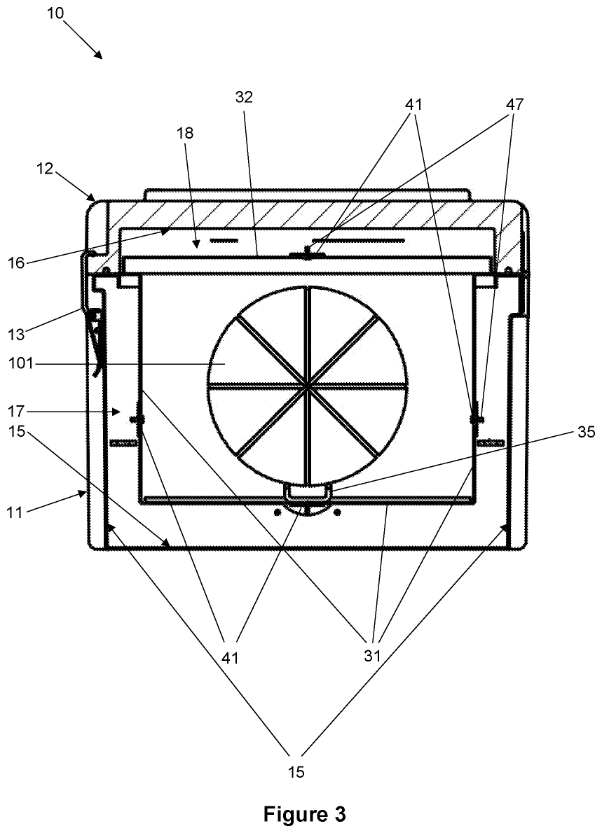

With reference to FIGS. 1 to 3, there is shown a diagrammatic view of a disinfecting container 10 in use according to a preferred embodiment. The disinfecting container 10 includes a body 11 and a cover 12. FIG. 1 shows the disinfecting container 10 with cover 12 in the opened configuration. FIG. 2 shows the disinfecting container 10 with cover 12 in the closed configuration. FIG. 3 shows a cross-sectional side view of the disinfecting container 10 with cover 12 in the closed configuration. FIGS. 1 and 3 shows two pieces equipment (balls) 101 placed within the disinfecting container. FIGS. 2 and 3 show that in the closed configuration, cover 12 is secured by a lock 13 to seal the container.

With reference to FIGS. 1 and 3, the disinfecting container 10 also includes UV emitting lights 21 within the container. The UV emitting lights 21 used are UV emitting LEDs (ZEUBE265-2CA, 265 nm wavelength, 50 mW light output). The UV emitting lights 21 are mounted to liners 31 of the body 11 and liner 32 of the cover 12. Mounting frames 41 are used to mount the UV emitting lights 21 to liners 31 and liner 32 by fasteners 47. Liners 31 and liner 32 have a plurality of first openings to accommodate the mounted UV emitting lights 21. Liners 31 and liner 32 also have a plurality of second openings to receive fasteners 47 for mounting the UV emitting lights 21 by mounting frames 41. The UV emitting lights 21 are powered by a power source (not shown) including batteries and AC/DC power source. There are two handles 14 located on opposite sides of the disinfecting container 10 (one of the handles is not shown) such that the disinfecting container is portable.

Liners 31 cover all of inner surfaces 15 of the body 11. There is a space 17 between liners 31 and inner surfaces 15 that allows air to flow through. The liners 31 form an internal recess within the body 11 where the pieces sporting equipment 101 is placed. Liner 32 covers inner surface 16 of the cover 12. Inner surface 16 is the side of cover 12 that is facing the interior of the body 11. There is a space 18 between the liner 32 and the inner surface 16 that allows air to flow through. The liners 31 and liner 32 are made of reflective material such that the disinfecting container 10 has an interior reflective surface.

With reference to FIG. 2, the disinfecting container 10 further includes a control system 52 on the cover 12. The control system 52 is operatively connected to a power source (not shown). The control system 52 is a touch screen digital display which turns on or off the UV emitting lights 21. The touch screen digital display is also displays the time remaining to complete a disinfection cycle and the status of the power source.

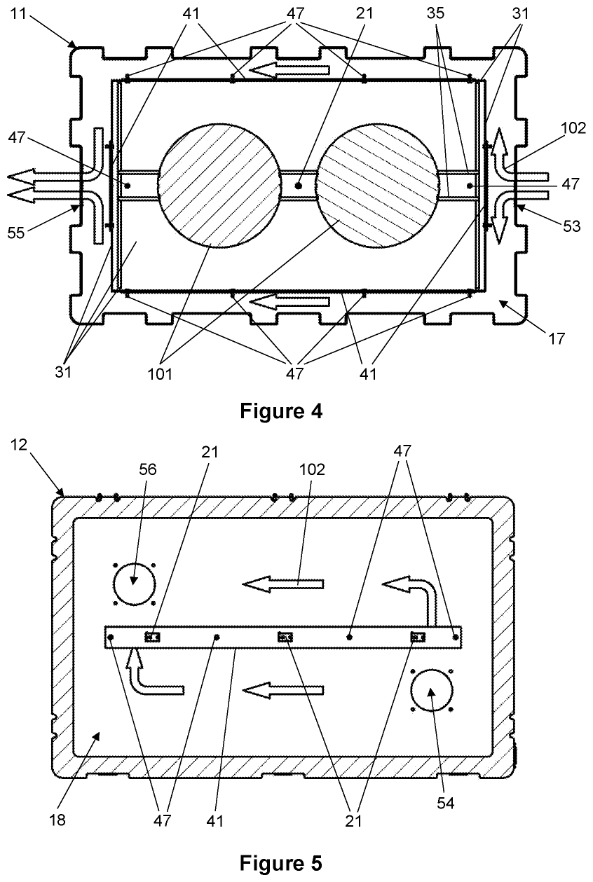

With reference to FIGS. 1, 2 and 4, the body 11 includes an air inlet 53 and an air outlet 55. Air 102 can flow from the outside through the air inlet 53 into space 17 within the body 11. The air 102 can then flow through the space 17 and out of the body 11 through air outlet 55. The air inlet 53 and air outlet 55 each further includes a HEPA filter. The HEPA filters at the air inlet 53 and air outlet 54 filter out particles and microorganisms from air 102 that is drawn through the body 11.

There is an exhaust fan located within the body 11 (not shown) that creates the air flow 102 in body 11. The airflow 102 in body 11 can help to reduce heat generated by the UV emitting lights 21 and other electronic components positioned within the body 11. The air flow 102 in body 11 can also help to dry the surface of the equipment placed within the disinfecting container.

With reference to FIGS. 1, 2 and 5, the cover 12 includes air inlet 54 and air outlet 56. Air 102 can flow from the outside through air inlet 54 into space 18 of the cover 12. The air 102 then flow from the space 18 and out of the cover 12 through air outlet 56. The air inlet 54 and air outlet 56 each further includes a HEPA filter. The HEPA filters at the air inlet 54 and air outlet 56 filter out particles and microorganisms from air 102 that is drawn through the cover 12.

There is an exhaust fan located within the cover 12 (not shown) that creates the air flow 102 in cover 12. The air flow 102 in cover 12 can help to reduce heat generated from the UV emitting lights 21 and other electronic components positioned at the cover 12.

With reference to FIGS. 1 to 5, in use, the pieces of sporting equipment 101 are placed on supporting rack 35 within the disinfecting container 10. Supporting rack 35 holds the pieces sporting equipment 101 up from the bottom of the body 11. The cover 12 is then closed and the disinfecting container is sealed by the lock 13.

A total of 14 UV emitting lights 21 are positioned within the disinfecting container so as to substantially irradiate the entire surface or exposed surface of the sporting equipment 101. There are three UV emitting lights 21 positioned at the cover 12, with one UV emitting light 21 being provided between two fasteners 47. The remaining UV emitting lights 21 are positioned at the body as followed: three UV emitting lights 21 provided on each of the front, bottom and back direction of the sporting equipment, and one UV emitting light 21 provided on opposite sides of the sporting equipment. The interior reflective surface of the container formed by the liners 31 and liner 32 reflects the UV radiation emitted from the UV emitting lights 21 such that the entire surface exposed surface of the sporting equipment is substantially irradiated.

To conduct a disinfection cycle, the UV emitting lights 21 are turned on by the control system 52. To achieve substantial disinfection, the UV emitting lights 21 emit UV radiation with an intensity of 0.05 to 0.06 mW/cm² and the disinfection cycle is set to complete within 76 seconds to 90 seconds. The UV radiation from the UV emitting lights 21 have a preferred wavelength in the range of substantially 200 to 280 nm, more preferably a wavelength of 265 nm. After the disinfection cycle is completed, the UV emitting lights are turned off by the control system 52.

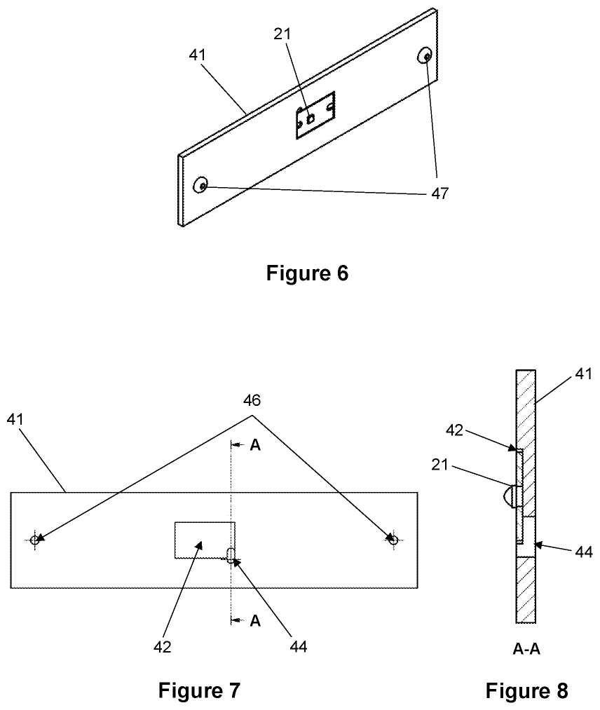

With reference to FIGS. 6 to 8, there is shown a mounting frame 41 according to a preferred embodiment. FIG. 8 shown the view of cross-section A-A in FIG. 7. The mounting frame 41 includes a pocket 42 to house the UV emitting light 21, and a mounting aperture 44 for attaching and detaching the UV emitting light 21 from pocket 42. The mounting frame 41 also include mounting openings 46 for receiving fastener 47 when mounting to the liners 31 and liner 32.

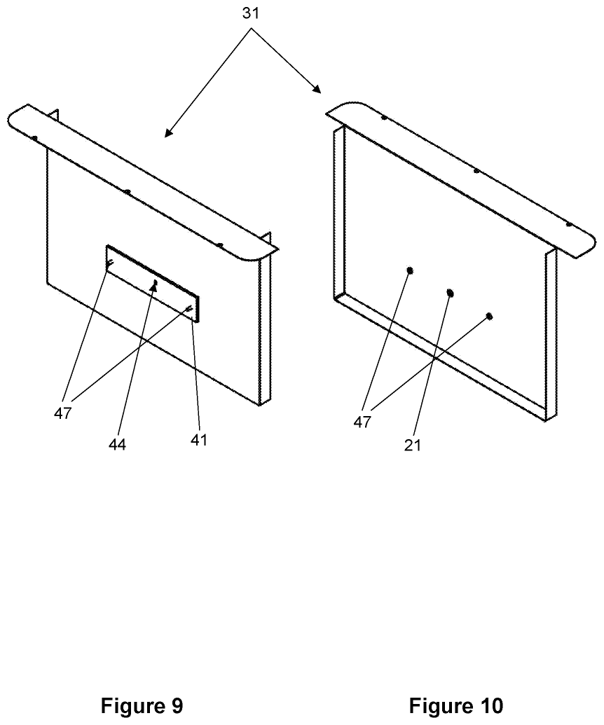

With reference to FIGS. 9 and 10, there is shown the front and back view of the liner 31 with the mounting frame 41, respectively. In use, the UV emitting light 21 sits within pocket 42 (not shown) and is attached to the mounting frame 41 through the mounting aperture 44. The mounting frame 41 is then mount on the back side of liner 31 and is secured by fasteners 47. Liner 31 has a first openings to accommodate the mounted UV emitting light 21 such that the UV emitting light is visible on the front side of liner 31. Liner 31 also had a plurality of second openings to receive fasteners 47 that also passes through to the corresponding mounting openings 46 on the mounting frame 41. The fasteners 47 helps to secure the mounting frame 41 to the liner 31.

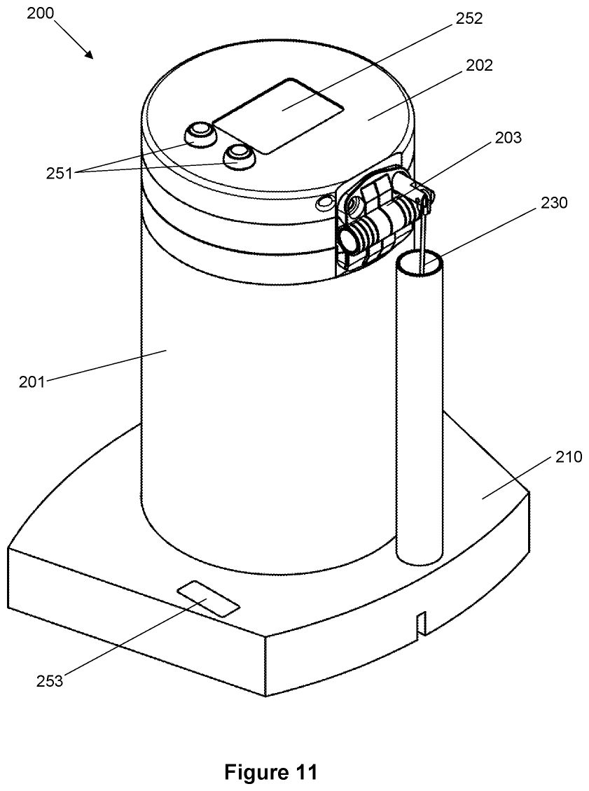

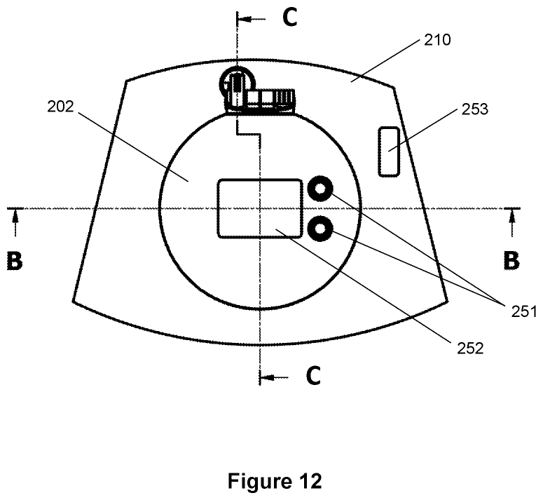

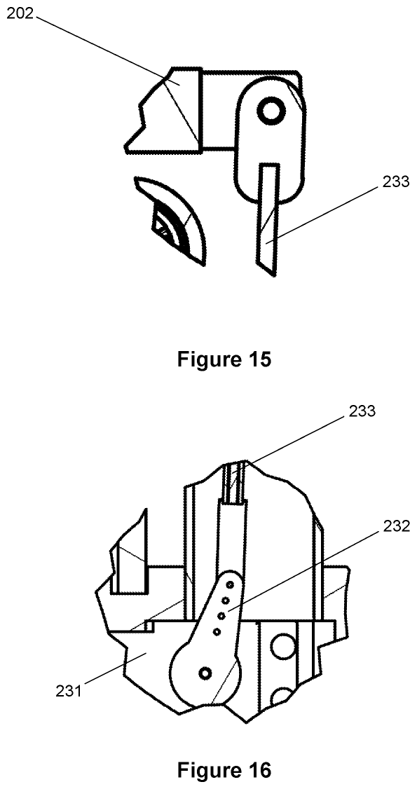

With reference to FIGS. 11 to 16 there is depicted a disinfecting container 200 in accordance with a further embodiment of the disclosure. The disinfecting container 200 includes a body 201 and a cover 202 pivotally connected to the body 201 by a hinge 203. The body 201 has a base 210 and a substantially transparent cylindrical housing 211 mounted on the base 210. The housing 211 having a recess.

The cover 202 is moveable between an open configuration allowing access to the interior space of the housing 211 and a closed configuration sealingly securing the cover 202 and the body 201. FIGS. 11 to 16 show the disinfecting container 200 with cover 202 in the closed configuration.

The disinfecting container 200 has an actuator 230 attached to the base 210 and the cover 202. Activation for the actuator 230 to moves the disinfecting container 200 between the open configuration and closed configuration.

The body 201 has an inner chamber 212 fitted within the recess of the housing 211. The inner chamber 212, base 210 and cover 202 define an interior space with an openable top. The surfaces of the interior space are reflective.

An array of eight UV emitting lights 221 are mounted to the inner chamber 212 and arranged to emit UV light into the inner chamber 212. The array of UV emitting lights 221 are positioned within the inner chamber 212 to substantially irradiate the entire surface or exposed surface of an object to be disinfected via direct UV light and UV light reflected from the surfaces of the interior space. The UV emitting lights 22 are positioned such that: one is centred on the bottom of the cover 202, one is centred on the top of the base 210, and six are placed around the inside of the inner chamber 212. The UV emitting lights 221 used are UV emitting LEDs (ZEUBE265-2CA, 265 nm wavelength, 50 mW light output). The UV emitting lights 221 are powered by a power source (not shown) including batteries and an AC/DC power source.

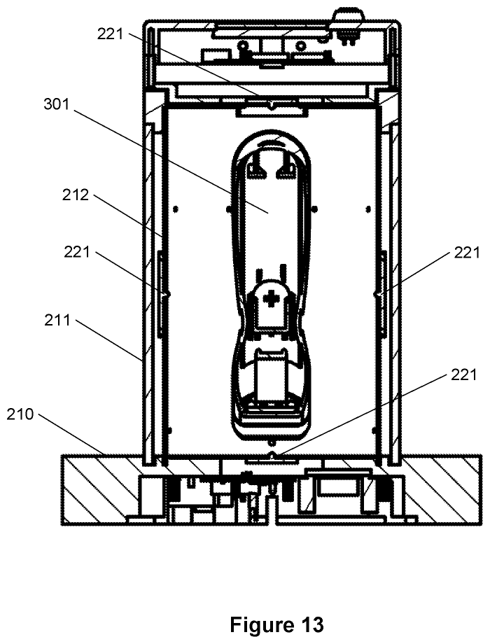

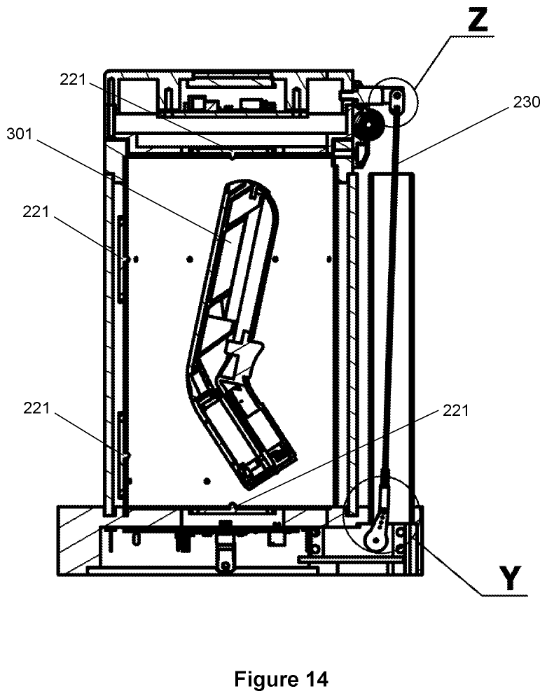

FIGS. 13 and 14 depict an object 301 to be disinfected placed within the inner chamber 212. The object 301 is suspended on wires (not shown).

The disinfecting container 200 has control system, located within the base 210 (not shown) and connected to a power source (not shown). The control system is configured to turn on and off the UV emitting lights 221 and run a disinfection cycle for a predetermined period. The control system includes human interface controls 251 and a display 252 on the cover 202. The display 252 displays the time remaining to complete a disinfection cycle and the status of the power source.

The control system may have one or more pre-set disinfection cycles selectable for different circumstances. For example, a short cycle is required for viral only decontamination, and a long cycle is required for full viral and bacterial decontamination.

The actuator 230 is controllably connected to the control system. The actuator 230 comprises a servo 231 mounted within a recess of the base 210 connected pivotally via a servo arm 232 to one end of a rod 233. Another end of the rod 233 is pivotally connected to the cover 202 and arranged such that lowering the rod 233 pivotally raises the cover 202. The control system can activate the actuator 230 to raise the cover 202 such that the disinfecting container 200 is in the open configuration upon finishing a disinfection cycle.

The control system includes a motion sensor 253 on the base 210 to sense a gesture and action the actuator 230. The control system commences a disinfection cycle upon motion sensor 253 sensing a gesture to close the cover 202.

The container 200 has a plurality of LED indicators mounted within the housing 211 and observable through the transparent housing 211 and indicate the status (off/on/finished) of the disinfecting cycle.

To conduct a disinfection cycle, the UV emitting lights 221 are turned on by the control system. To achieve substantial disinfection, the UV emitting lights 221 emit UV radiation with an intensity of 0.05 to 0.06 mW/cm2 and the disinfection cycle is set to complete within 76 seconds to 90 seconds. The UV radiation from the UV emitting lights 21 has a wave length of 265 nm. After the disinfection cycle is completed, the UV emitting lights are turned off by the control system.

In use, the disinfecting container 200 is opened by performing a gesture above the motion sensor 253, placing an object inside the inner chamber 212, closing and initiating a disinfection cycle the preforming another gesture above the motion sensor 253, performing a disinfection cycle, automatically transitioning the disinfecting container 200 to the open configuration upon completion of the disinfection cycle so that the object can be removed.

### EXAMPLES

The device used to disinfect the test objects was the device described in at least one embodiment of the present disclosure.

### Example 1—Virus

### Virus Strains

The three test viruses used in this study Polio ATCC/VR 192, Herpes Simplex (HSV-1) ATCC/VR 733 and Murine hepatitis (MHV-1) ATCC/VR 261 were obtained from the American Type Culture Collection (ATCC). The viruses were stored in liquid nitrogen prior to use.

### Cell Substrates

The two host cells used in this study Vero (ATCC/CCL-81) and A9 (ATCC/CCL-1.4), were obtained from the ATCC. The host cells were stored in liquid nitrogen prior to use. Cells were thawed and sub-cultured in MEM cell growth medium.

### Test Materials

I. Carrier made from Sport's ball material (5×5 cm)
II. 2×Footballs each fitted with 2 Alligator clips

### Preparation of Cell Substrates:

1. All work was carried out in a class 2 biosafety cabinet.
2. Growth media was prepared by combining the following reagents in MEM in their specified proportions; FBS (10%), L-glutamine (1%), Antibiotic-Antimycotic (1%) and HEPES Buffer (1%).
3. Two 25 cm² flasks were prepared containing monolayers of Vero (ATCC/CCL-81) and A9 (ATCC/CCL 1.4) cells.
4. Once the monolayers reached confluency, the growth media was discarded and each cell monolayer was washed twice with 2.0 mL PBS, and the remaining PBS being was discarded.
5. A volume of 0.5 mL Trypsin was then added to each flask, which were subsequently incubated at 37° C.±2° C. for approximately 5 min until cells were visibly lifting from the flasks. Progress was checked using an inverted microscope.
6. When all the cells were detached, 5.0 mL of growth media was added to each flask and the flasks were shaken gently to resuspend the cells.
7. A volume of 1.0 mL of the cell suspension was removed and diluted in 10.0 mL of fresh growth media. Using a multichannel pipette, 0.1 mL of this cell suspensions was dispensed into each well of a 96 well microtiter plate.
8. The plates were incubated in the CO₂ incubator with an atmosphere of 5% CO₂ in air at a temperature of 37° C. 2° C. for 18-24 hr.

### Preparation of Virus

1. Three virus vials, Polio, HSV 1 and MHV 1 were removed from liquid nitrogen storage and thawed in a 37° C.±1° C. water bath for approximately 5 min.
2. Maintenance medium (MM) was prepared by combining the following reagents in MEM in their specified proportions; FBS (2%), L-glutamine (1%), Antibiotic-Antimycotic (1%) and Hepes Buffer (1%). The MM was used as diluents for preparing virus dilutions.
3. The initial stock viruses were diluted in series from 10⁻¹ to 10⁻⁹.
4. Infectivity was assayed by plating out an aliquot of 100 μL of each dilution into quadruplicate wells of the prepared cell substrates (Table 1).

TABLE 1.1
Cell substrates for the
test viruses for the study

| Test Virus | Cell Substrate |
|---|---|
| Murine hepatitis virus | A9 Cells ATCC/CCL 1.4 |
| (MHV1: ATCC/VR261) | |
| Polio Virus ATCC/VR 192 | Vero cells ATCC/CCL 81 |
| Herpes Simplex Virus | Vero cells ATCC/CCL 81 |
| ATCC/VR 733 | |

### Preparation of Virus Control Carrier

1. A volume of 0.2 mL of each stock virus was inoculated into three test carriers and three positive control carriers (one carrier per each virus). The virus suspensions were evenly distributed by using a sterile plastic spreader and allowed to dry.
2. Three positive controls carriers were placed into the 70 mL sterile container containing 2 mL MM and vortexed for 2 min.
3. The solutions were then serially diluted and plated out onto prepared cell monolayers in quadruplicate wells of the prepared cell substrates.

### Disinfectant Efficacy Study

1. The first Football was used for two pre-inoculated carriers with two different viruses (Polio and HSV-1). The inoculated test carriers were attached to the Football using the Alligator clips.
2. The second Football was used for the carrier inoculated with MHV-1 and one negative, un-inoculated carrier.
3. The Device was run as per Manufacturer's instruction (Appendix 3 of approved protocol NJ21AB0206-1).
4. At the end of the run, the carriers were retrieved by placing them into three sterile 70 mL container containing 2.0 mL of MM, one container for each virus and vortexed for 2 min.
5. The solutions were then serially diluted in MM and plated out onto the prepared cell monolayers in quadruplicate.

### Preparation of Cytotoxicity Control

6. An aliquot of 0.2 mL of MM was inoculated onto the 3 carriers and using sterile spreader and allowed to dry.
7. Place the carriers into the position as described in H.1 and run the device.
8. At the end of the run, transferred each carrier into sterile 70 mL containers containing 2.0 mL MM and vortexed for 2 min.
9. 0.2 mL of the liquid was serially diluted in 1.8 mL maintenance media containing 2% FBS up to 10⁻³.
10. Infectivity was assayed by plating out an aliquot of 100 μL of each dilution into quadruplicate wells.

### Preparation of Neutralisation Control

11. Dilutions of the neutralised sample filtrates from (10⁻² to 10⁻³) of the Cytotoxicity control (I. 4) were spiked with 0.1 mL of low titre of virus suspension.
12. A volume of 0.1 mL of spiked neutralisation dilutions were plated out in quadruplicate along with the test and virus control plate.
13. The cell infectivity dilution(s) was (were) noted and indicated that the sample had been completely neutralized.

### Preparation of Negative Control

14. A sterile, un-inoculated carrier was clipped on to the Football using an Alligator clip and placed inside the Device (H.2).
15. Device was run as per the Manufacturer's instruction.
16. At the end of the run, the negative control carrier was transferred into a sterile 70 mL container containing 2 mL MM and vortexed for 2 min.
17. Negative control eluents were plated out 0.1 mL in quadruplicate into the cell substrate.

### Method for Viral Assay/Infectivity

18. Commencing with the highest dilution of the Stock virus (F), Positive control (G), Test carrier (H), Cytotoxicity control (I), Neutralisation control (J) and Negative controls (K), 100 L from each dilution was dispensed into quadruplicate wells of the prepared cell substrates (E).
19. The seeded plates were incubated at 37° C.±2° C. in a humidified atmosphere of 5%-7% CO2 for 7 days.

### Virus Titrations

20. The test plates were periodically examined over seven days for presence/absence of cytopathic effect (CPE) and cytotoxicity.
21. The CPE effect at each well of dilution of positive, negative, cytotoxicity, neutralisation and test was recorded.
22. The Reed & Muench LD50 Method was used to determine the virus titre endpoint.

### Results

Results are presented in Tables 2, 3 and 4. Log reductions are presented in Table

5. The Device showed no cytotoxicity at the 1:10 dilution (less than 1.0 log₁₀). In addition, the neutralisation control shows no viral inhibition at 10⁻¹ dilution, which means the sample was neutralised less than 1.0 log₁₀. There was no CPE was observed for the negative control.

TABLE 1.2
Results for MHV-1

| | | | Virus | | | | | |
|---|---|---|---|---|---|---|---|---|
| Virus | Stock | Percentage | Control | Percentage | | | Test | Percentage |
| Dilution | virus | (%) | Carrier | (%) | Cytotoxicity | Neutralisation | result | (%) |
| 10⁻¹ | 4⁺/4 | 100.00 | 4⁺/4 | 100.00 | 0/4 | 4/4 | 1⁺/4 | 25.00 |
| 10⁻² | 4⁺/4 | 100.00 | 4⁺/4 | 100.00 | 0/4 | 4/4 | 0⁺/4 | 0.00 |
| 10⁻³ | 4⁺/4 | 100.00 | 4⁺/4 | 100.00 | 0/4 | 4/4 | 0⁺/4 | 0.00 |
| 10⁻⁴ | 4⁺/4 | 100.00 | 4⁺/4 | 100.00 | N/A | N/A | 0⁺/4 | 0.00 |
| 10⁻⁵ | 4⁺/4 | 100.00 | 4⁺/4 | 100.00 | N/A | N/A | 0⁺/4 | 0.00 |
| 10⁻⁶ | 4⁺/4 | 100.00 | 2⁺/4 | 66.67 | N/A | N/A | 0⁺/4 | 0.00 |
| 10⁻⁷ | 4⁺/4 | 100.00 | 2⁺/4 | 33.33 | N/A | N/A | 0⁺/4 | 0.00 |
| 10⁻⁸ | 4⁺/4 | 100.00 | 0⁺/4 | 0.00 | N/A | N/A | 0⁺/4 | 0.00 |
| Total | 32 | N/A | 24 | N/A | N/A | N/A | 32 | N/A |
| host | | | | | | | | |
| Log₁₀ | >8.50 | N/A | 6.50 | N/A | <1.00 | <1.00 | <1.00 | N/A |

| | |
|---|---|
| Log₁₀ Reduction of Virus after treatment | >5.50 |

TABLE 1.3
Results for HSV-1

| | | | Virus | | | | | |
|---|---|---|---|---|---|---|---|---|
| Virus | Stock | Percentage | Control | Percentage | | | Test | Percentage |
| Dilution | virus | (%) | Carrier | (%) | Cytotoxicity | Neutralisation | result | (%) |
| 10⁻¹ | 4⁺/4 | 100.00 | 4⁺/4 | 100.00 | 0/4 | 4/4 | 0⁺/4 | 0.00 |
| 10⁻² | 4⁺/4 | 100.00 | 4⁺/4 | 100.00 | 0/4 | 4/4 | 0⁺/4 | 0.00 |
| 10⁻³ | 4⁺/4 | 100.00 | 4⁺/4 | 100.00 | 0/4 | 4/4 | 0⁺/4 | 0.00 |
| 10⁻⁴ | 4⁺/4 | 100.00 | 4⁺/4 | 100.00 | N/A | N/A | 0⁺/4 | 0.00 |
| 10⁻⁵ | 4⁺/4 | 100.00 | 4⁺/4 | 100.00 | N/A | N/A | 0⁺/4 | 0.00 |
| 10⁻⁶ | 4⁺/4 | 100.00 | 4⁺/4 | 100.00 | N/A | N/A | 0⁺/4 | 0.00 |
| 10⁻⁷ | 4⁺/4 | 100.00 | 4⁺/4 | 100.00 | N/A | N/A | 0⁺/4 | 0.00 |
| 10⁻⁸ | 4⁺/4 | 100.00 | 1⁺/4 | 25.00 | N/A | N/A | 0⁺/4 | 0.00 |
| Total | 32 | N/A | 29 | N/A | N/A | N/A | 32 | N/A |
| host | | | | | | | | |
| Log₁₀ | >8.50 | N/A | 7.67 | N/A | <1.00 | <1.00 | <1.00 | N/A |

| | |
|---|---|
| Log₁₀ Reduction of Virus after treatment | >6.67 |

TABLE 1.4
Results for Poliovirus

| | | | Virus | | | | | |
|---|---|---|---|---|---|---|---|---|
| Virus | Stock | Percentage | Control | Percentage | | | Test | Percentage |
| Dilution | virus | (%) | Carrier | (%) | Cytotoxicity | Neutralisation | result | (%) |
| 10⁻¹ | 4⁺/4 | 100.00 | 4⁺/4 | 100.00 | 0/4 | 4/4 | 4⁺/4 | 100.00 |
| 10⁻² | 4⁺/4 | 100.00 | 4⁺/4 | 100.00 | 0/4 | 4/4 | 4⁺/4 | 100.00 |
| 10⁻³ | 4⁺/4 | 100.00 | 4⁺/4 | 100.00 | 0/4 | 4/4 | 1⁺/4 | 25.00 |
| 10⁻⁴ | 4⁺/4 | 100.00 | 4⁺/4 | 100.00 | N/A | N/A | 0⁺/4 | 0.00 |
| 10⁻⁵ | 4⁺/4 | 100.00 | 4⁺/4 | 100.00 | N/A | N/A | 0⁺/4 | 0.00 |
| 10⁻⁶ | 4⁺/4 | 100.00 | 4⁺/4 | 100.00 | N/A | N/A | 0⁺/4 | 0.00 |
| 10⁻⁷ | 4⁺/4 | 100.00 | 4⁺/4 | 100.00 | N/A | N/A | 0⁺/4 | 0.00 |
| 10⁻⁸ | 4⁺/4 | 100.00 | 4⁺/4 | 100.00 | N/A | N/A | 0⁺/4 | 0.00 |
| Total | 32 | N/A | 32 | N/A | N/A | N/A | 32 | N/A |
| host | | | | | | | | |
| Log₁₀ | >8.50 | N/A | >8.50 | N/A | <1.00 | <1.00 | 2.67 | N/A |

| | |
|---|---|
| Log₁₀ Reduction of Virus after treatment | >5.83 |

TABLE 1.5
Summary of log reduction

| Virus Titre | | | |
|---|---|---|---|
| (Log10) | MHV-1 | HSV-1 | Poliovirus |
| Stock virus | >8.50 | >8.50 | >8.50 |
| Positive control | 6.50 | 7.67 | >8.50 |
| Test carrier | <1.00 | <1.00 | 2.67 |
| Log reduction | >5.50 | >6.67 | >5.83 |
| Percentage | >99.9997 | >99.9999 | >99.9999 |
| reduction (%) | | | |

Note:
Presence of virus in each response is recorded as “+”
Absence of virus in each response is recorded as “0”
Cytotoxic response is recorded as “C”
Calculated MHV-1 stock titre (0 hr) = 10^{>8.50} TCID_{50/0.1 ml} (>8.50 log₁₀)
Calculated HSV-1 stock titre (0 hr) = 10^{>8.50} TCID_{50/0.1 ml} ((>8.50 log₁₀)
Calculated Polio virus stock titre (0 hr) = 10^{>8.50} TCID_{50/0.1 ml} ((>8.50 log₁₀)
Cell control—4 wells with healthy cell monolayer
*The Reed & Muench LD50 Method was used for determining the virus titre endpoint.

### Conclusions:

Considering the cytotoxicity and neutralization test results, the sample has shown virucidal efficacy against MHV1, HSV1 and Polio by achieving greater than 5.50, 6.67 and 5.83 log reduction respectively in virus concentration after a 90 Seconds cycle at room temperature under clean conditions.

### Example 2—Bacteria

TABLE 2.1
Test Conditions

| | |
|---|---|
| Test Condition | Dirty (0.3% BSA) |
| Neutraliser | T6 (10 mL) |
| Operating Cycle | 90 Seconds |

TABLE 2.2
Neutralisation Validation

| | | | Method |
|---|---|---|---|
| | Control | Neutraliser | Validation |
| Organism | Count (Nv) | Control (NC) | (NT) |
| S. aureus | 82 | 86 | 81 |
| ATCC 6538 | | | |
| E. coli | 63 | 68 | 67 |
| ATCC 10536 | | | |
| P. aeruginosa | 59 | 55 | 52 |
| ATCC 15442 | | | |
| E. hirae | 37 | 32 | 34 |
| ATCC 10541 | | | |
| MRSA | 35 | 34 | 32 |
| ATCC 33591 | | | |

| | |
|---|---|
| Negative | No Growth |
| Control | |

TABLE 2.3
Bactericidal Test Result

| | Recovered Count | | |
|---|---|---|---|
| | CFU/mL (log₁₀) | | |
| | Positive | | Log |
| Organism | Control | Test Result | Reduction |
| S. aureus | 9.91 × 10⁶ | 2.36 × 10⁵ | 1.63 |
| ATCC 10536 | (7.00) | (5.37) | |
| E. coli | 1.01 × 10⁷ | 6.85 × 10⁴ | 2.16 |
| ATCC 10536 | (7.00) | (4.84) | |
| P. aeruginosa | 5.45 × 10⁶ | 4.90 × 10⁵ | 1.05 |
| ATCC 15442 | (6.74) | (5.69) | |
| E. hirae | 9.85 × 10⁶ | 2.82 × 10⁶ | 0.54 |
| ATCC 10541 | (6.99) | (6.45) | |
| MRSA | 7.90 × 10⁶ | 1.68 × 10⁵ | 1.67 |
| ATCC 33591 | (6.90) | (5.23) | |

CFU = Colony Forming Units

Comments: The product achieved 1.63 log reduction against S. aureus, 2.16 log reduction against E. coli, 1.05 log reduction against P. aeruginosa, 0.54 log reduction against E. hirae, and 1.67 log reduction against MRSA when tested as per conditions above.

### Example 3—Bacteria

TABLE 3.1
Test Conditions

| | |
|---|---|
| Test Condition | Dirty (0.3% BSA) |
| Neutraliser | T6 (10 mL) |
| Operating Cycle | 4 Minutes |

TABLE 3.2
Neutralisation Validation

| | Control | Neutraliser | Method |
|---|---|---|---|
| | Count | Control | Validation |
| Organism | (Nv) | (NC) | (NT) |
| S. aureus | 49 | 39 | 48 |
| ATCC 6538 | | | |
| E. coli | 32 | 36 | 35 |
| ATCC 10536 | | | |

| | |
|---|---|
| Negative | No |
| Control | Growth |

CFU = Colony Forming Unit

TABLE 3.3
Bactericidal Test Result

| | Recovered Count | | |
|---|---|---|---|
| | CFU/mL (log₁₀) | | |
| Organism | Positive Control | Test Result | Log Reduction |
| S. aureus | 9.80 × 10⁷ | 5.40 × 10² | 5.26 |
| ATCC 6538 | (7.99) | (2.73) | |
| E. coli | 3.02 × 10⁷ | 1.36 × 10⁴ | 3.35 |
| ATCC 10536 | (7.48) | (4.13) | |

CFU = Colony Forming Unit

Comments: The product achieved 5.26 log reduction against S. aureus, and 3.35 log reduction against E. coli when tested as per conditions above.

It should be appreciated that embodiments of the present disclosure provide an advantage of an effective disinfection treatment to the surface of the personal goods and equipment that is quick and easy. Embodiments of the present disclosure provide an advantage in that the whole process of disinfection can be completed in substantially two minutes or less. Embodiments of the present disclosure also provide an advantage of a reproducible disinfection treatment for the personal goods and equipment. Embodiments of the present disclosure further provide an advantage of reducing human error in performing the treatment of disinfecting goods and equipment. Embodiments of the present disclosure also provide an advantage of substantially drying the surface of the equipment. Embodiments of the present disclosure further provide an advantage of reducing damage to the outer material of the equipment caused by to physical treatment. Embodiments of the present disclosure also provide an advantage of a disinfecting container that is portable and movable. Embodiments of the present disclosure further provide an advantage of a mobile disinfecting container that can be self-powered by batteries or powered by the main power source.

Embodiments of the present disclosure provide an advantage of an effective disinfection treatment to the surface of small, particularly handheld, personal goods and equipment that is quick and easy. Embodiments of the present disclosure provide an advantage in that the whole process of disinfection can be completed in substantially two minutes or less. Embodiments of the present disclosure also provide an advantage of a reproducible disinfection treatment for the personal goods and equipment. Embodiments of the present disclosure further provide an advantage of reducing human error in performing the treatment of disinfecting goods and equipment. In particular, the controllable mechanism for opening and closing the cover, and automation thereof reduces the likelihood of misuse. Embodiments of the present disclosure further provide an advantage of reducing damage to the outer material of the equipment caused by physical treatment. Embodiments of the present disclosure also provide an advantage of a further container that is portable and movable. Embodiments of the present disclosure further provide an advantage of a mobile disinfecting container that can be self-powered by batteries or powered by the main power source. Embodiments of the present disclosure further provide an advantage of selectable pre-set disinfection cycles selectable dependent on whether a short cycle is required for viral only decontamination, or a long cycle is required for full viral and bacterial decontamination.

The gesture-based activation of the present embodiment advantageously reduces the amount of contact with the disinfecting container, particularly the commonly handled controls of the disinfecting container. In situations where multiple contaminated objects are being disinfected in succession, the reduction of the need to physically touch the disinfecting container reduces the possibility of cross-contamination between disinfection procedures.

It will of course be realized that while the foregoing has been given by way of illustrative example of this invention, all such and other modifications and variations thereto as would be apparent to persons skilled in the art are deemed to fall within the broad scope and ambit of this invention as is herein set forth.

Throughout the description and claims of this specification the word “comprise” and variations of that word such as “comprises” and “comprising”, are not intended to exclude other additives, components, integers or steps.

1. A disinfecting container for treating a good or piece of equipment, the container including:
a body in which to place the good or piece of equipment, the body has an internal recess and the internal recess has an interior reflective surface;
a cover that is attached or attachable to the body;
at least one UV emitting light within the container; and
at least one control system that is operatively associated with a power source, the control system is configured to turn on or off the at least one UV emitting light,
wherein the UV emitting light emits UV at a wave length and for a predetermined period to achieve disinfection on a surface of the good or piece of equipment.

2. A disinfecting container according to claim 1, wherein the at least one UV emitting light preferably emits UV that has a wavelength in the range of 250 to 265 nm.

3. A disinfecting container according to claim 1, wherein the container includes a plurality of UV emitting lights arranged and positioned in a pattern that irradiates the entire surface or exposed surface of the surface of the good or piece of equipment placed within the container.

4. A disinfecting container according to claim 1, wherein the container includes a plurality of UV emitting lights, each UV emitting light is a UV light-emitting diode (LED), and wherein at least one UV emitting light is positioned at the cover and at least one UV emitting light is positioned within the body of the container.

5. A disinfecting container according to claim 1, wherein the control system includes a safety switch configured to turn off the at least one UV emitting light when the cover is displaced from the container.

6. A disinfecting container according to claim 1, wherein the control system displays a status of the power source including whether or not there is sufficient power from the power source and/or the amount of power remaining from the power source.

7. A disinfecting container according to claim 1, wherein the container includes at least one liner formed by a reflective material that covers at least one inner surface of the container.

8. A disinfecting container according to claim 1, wherein the container includes at least one liner formed by a reflective material that reflects the UV radiation emitted from the UV emitting lights so that 90% or more of the surface of the good or piece of equipment is irradiated.

9. A disinfecting container according to claim 1, wherein the control system includes a UV sensor to gather readings of UV activity within the container during a disinfection cycle, and wherein the control system varies the time of UV light exposure required to complete a disinfection cycle based on the readings of the UV sensor.

10. A disinfecting container according to claim 1, wherein the disinfecting container includes at least one air inlet and at least one air outlet, the at least one air inlet and at least one air outlet allows air flow through the container.

11. A disinfecting container according to claim 1, wherein the disinfecting container includes at least one air inlet, at least one air outlet, and one or more extractors to create an air flow within the disinfecting container, wherein the one or more extractors extractor draws air through the at least one air inlet and out through the at least one air outlet.

12. A disinfecting container according to claim 1, wherein the container includes a dehumidifier to dry a wet object placed within the container.

13. A disinfecting container according to claim 1, wherein the disinfecting container includes at least one air inlet, at least one air outlet, and one or more extractors to create an air flow within the disinfecting container, the at least one air inlet and or the at least one air outlet has at least one air filter to filter particles or microorganisms.

14. A disinfecting container according to claim 1, wherein the disinfecting container includes at least one support that holds the good or piece of equipment in position within the body.

15. A disinfecting container according to claim 1, wherein the intensity of the UV radiation on the surface of the good or piece of equipment is in the range of 0.05 to 0.1 mW/cm².

16. A disinfecting container according to claim 1 wherein, the control system is configured to turn on the at least one UV emitting light when the container is in a closed configuration and an object is within the container and turn off the at least one UV emitting light after a predetermined period.

17. A disinfecting container for treating a good or piece of equipment, the container including:
a body in which to place the good or piece of equipment, the body has an internal recess and the internal recess has an interior reflective surface;
a cover that is attached or attachable to the body;
at least one array of UV emitting lights within the container;
at least one control system that is operatively associated with a power source, the control system is configured to turn on or off the at least one array of UV emitting lights;
wherein the at least one array of UV emitting lights emits UV light at a wavelength in the range of 250 to 265 nm;
wherein each UV emitting light has an angle of emission of 120 degrees;
wherein each UV emitting light arranged such that the emitted light travels within a range of 40 mm to 140 mm from the UV emitting light to the object; and
wherein the arrangement of array of UV emitting lights achieves a UV radiation intensity of at least 4.5 mJ/cm² on the surface of the good or piece of equipment placed within the container and for a predetermined period to achieve disinfection on the surface of the good or piece of equipment.
